(19) United States
(12) Patent Application Publication
(10) Pub. No.: US 2008/0275454 A1
(43) Pub. Date: Nov. 6, 2008

(54) Lumbar pedicular-facet fixation system and instrumentation

(76) Inventor: Paul T. Geibel, San Antonio, TX (US)

Correspondence Address: JACKSON WALKER LLP, 901 MAIN STREET, SUITE 6000, DALLAS, TX, 75202-3797, US

(21) Appl. No.: 12/151,164

(22) Filed: May 5, 2008

Related U.S. Application Data
(60) Provisional application No. 60/927,573, filed on May 4, 2007.

(51) Int. Cl. A61B 17/58; A61F 5/00

(52) U.S. Cl. 606/96; 606/86 R

(57) ABSTRACT

An implant and surgical system designed to guide and promote segmental instrumentation and stabilization through a pediculo-facet fixation technique. The implant varies in size, diameter and attachments for maximal fixation across this axis. A targeting device and surgical technique for placement of said implants through an open or minimally invasive approach is described. The combination of surgical technique, targeting device and low profile implants reduces associated morbidity associated with many fusion procedures.

### RELATED APPLICATIONS

This application claims priority to U.S. Provisional Patent Application, Ser. No. 60/927,573, entitled “Lumbar Pedicular-Facet Fixation System and Instrumentation” filed on May 4, 2007, having Paul Geibel listed as the inventor, the entire content of which is hereby incorporated by reference.

### FIELD OF THE INVENTION

The current invention relates generally to a lumbar pedicular-facet system in connection with spinal surgery, and more specifically to devices and methods useful for lumbar spinal fixation surgery.

### BACKGROUND

### I Spine Anatomy

The spine is made up of vertebrae connected sequentially to one another by posterior facet joints and discs of cartilage (invertabral discs). The facet joints and discs cause the spine to be flexible.

The spine is divided into four areas, the cervical (neck), thoracic (chest/trunk), lumbar (low back), and sacral (pelvic) areas. In addition, the spine has four characteristic curves, the cervical lordosis, thoracic kyphosis, lumbar lordosis, and sacral kyphosis.

### II Spinal Fixation Surgery

A number of pathological conditions involving the spine may result in discomfort, limited range of spinal motion, or potential damage to the nervous or circulatory system. In some cases, spinal fixation surgery can be performed to fix two or more vertebrae relative to one another, which may be effective in improving patient comfort. Examples of spinal pathologies which may be treatable using spine fusion surgery are degenerative disc disease; isthmic, degenerative, or postlaminectomy spondylolisthesis; a weak or unstable spine (caused by infections or tumors); fractures; scoliosis; or deformity.

In general, spinal fixation surgery may involve steps such as removing the lamina, or portion of the vertebra that covers the spinal cord, removing fractured or damaged elements of the vertebrae, introducing bone graft, and introducing a spinal fixation system to the affected vertebrae. A spinal fixation system may comprise screws or implants which attach to or interact with various elements of the vertebrae, or rods or implants held in place by the screws.

A variety of spinal fixation systems is available, including anterior systems which attach to the anterior portion of the spine, lateral systems which attach to the lateral portion of the spine, and posterior systems, which include rods attached to the vertebrae by either hooks attached to the lamina or transverse process, or screws inserted into the pedicles of the vertebrate.

A bone graft may also be introduced between two vertebrate. This can be either an autograft (often taken from the patient's hip), an allograft (often from donated or cadaver bone), or a synthetic bone graft substitute. Bone graft allows the development of a fused bone between the vertebrae, which fixes them relative to one another.

Because of the distinct anatomies of the vertebrae in the different areas of the spine, different spinal fusion surgical techniques have been developed for each area.

### III Methods of Spinal Fixation Surgery

Spinal fixation surgery was first described in the 1940 's, and there have since been a number of methods and devices described for carrying out related techniques.

U.S. Pat. No. 5,000,165 to Watanabe describes a lumbar spine fixation system which includes two spaced lumbar rods.

U.S. Pat. No. 5,584,887 to Kambin describes a method for percutaneous fixation of a pair of vertebrae of a patient.

U.S. Pat. No. 5,480,440 to Kambin describes a method for vertebral fixation of a pair of vertebrae of a patient.

U.S. Pat. No. 5,643,259 to Sasso describes spine fixation instrumentation.

US 2005/0149021 to Tozzi describes a spinal implant device designed to mimic and restore normal human spinal anatomy.

U.S. 2006/0235388 to Justis describes a system for augmenting a spinal joint.

U.S. 2008/0021473 to Butler describes pedicle screw constructs for spine fixation systems.

These earlier systems generally relied upon a system of rods and plates in order to fix vertebrae relative to one another. Optimal screw placement is not defined, making it difficult to drill and place implants at an angle resulting in maximum purchase.

Previous methods for internal fixation often involved screws which simply crossed from a superior to an anterior facet. The current method provides a more secure trajectory for an implant. The understanding of this trajectory, and the availability of an instrument to facilitate correct implant placement, will allow for more effective use of implants, and increased purchase in the bone, resulting in decreased levels of complications and morbidity

### SUMMARY

A preferred embodiment of the invention comprises a cannulated targeting device which may be used to guide a drill bit or implant at a standardized angle to the spinal bone surface to be drilled. This may cause the drill bit or implant to be placed at an angle of approximately 20-30 degrees medial to lateral (traveling away from the midline of the body), and approximately 30-40 degrees caudal inclination (traveling away from the head of the body) relative to the axis of the spine. This may further cause the drill bit or implant to follow a pathway from the inferior facet of an upper vertebra through a superior facet of a lower vertebra, through a pedicle, and optionally through a lateral pedicle wall, achieving high purchase relative to other possible methods of fixing bones.

A further preferred embodiment may comprise a method of fixing two vertebrae relative to one another comprising the steps of: a) drilling into the inferior facet of an upper vertebra at an angle of approximately 20-30 degrees medial to lateral (traveling away from the midline of the body), and approximately 30-40 degrees caudal inclination (traveling away from the head of the body); and b) removing the drill bit to form an opening; and c) inserting an implant into the opening formed by the drill; and d) fixing the implant in the opening to secure the vertebrae.

Yet another preferred embodiment may comprise an implant which may be used in spine fixation surgery to secure an inferior facet of an upper vertebra to a superior facet of a lower vertebra and to a pedicle, optionally projecting through a lateral pedicle wall.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows an upper and a lower vertebra in the spine. Asteriscks indicate possible points along the trajectory of a drill or implant in a preferred embodiment of the invention;

FIG. 2 shows the trajectory of a drill or implant in a preferred embodiment of the invention relative to an upper and a lower vertebra;

FIG. 3 shows a possible embodiment of a cannulated targeting device in accordance with the invention;

FIG. 4 shows vertebrae of the lumbar spine to be fixed in accordance with a preferred embodiment of the invention;

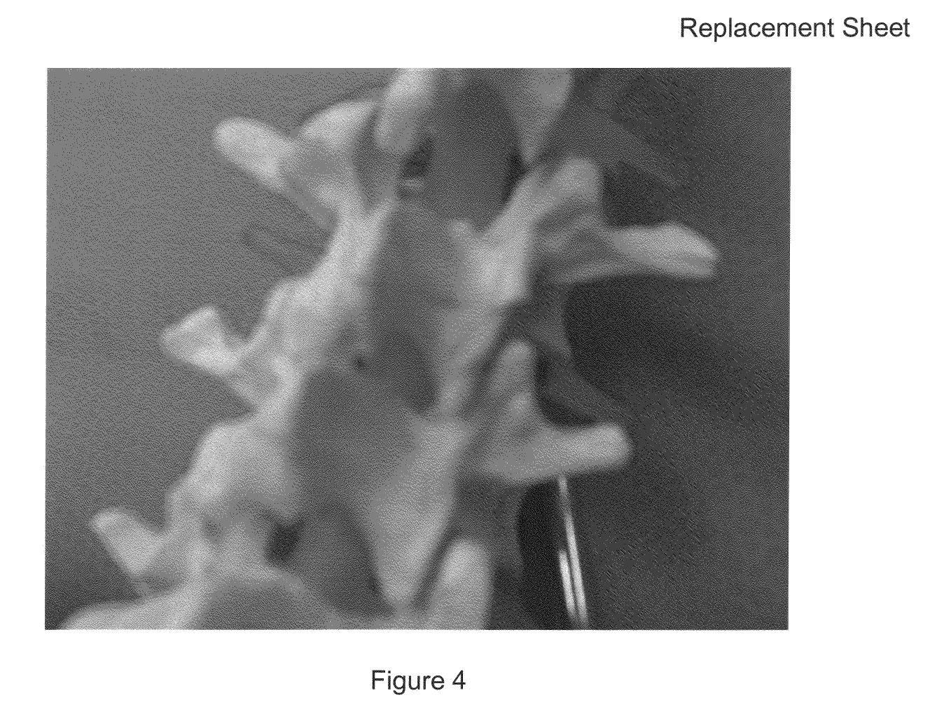

FIG. 5 shows a drill bit being placed for drilling into a vertebra in accordance with a preferred embodiment of the invention;

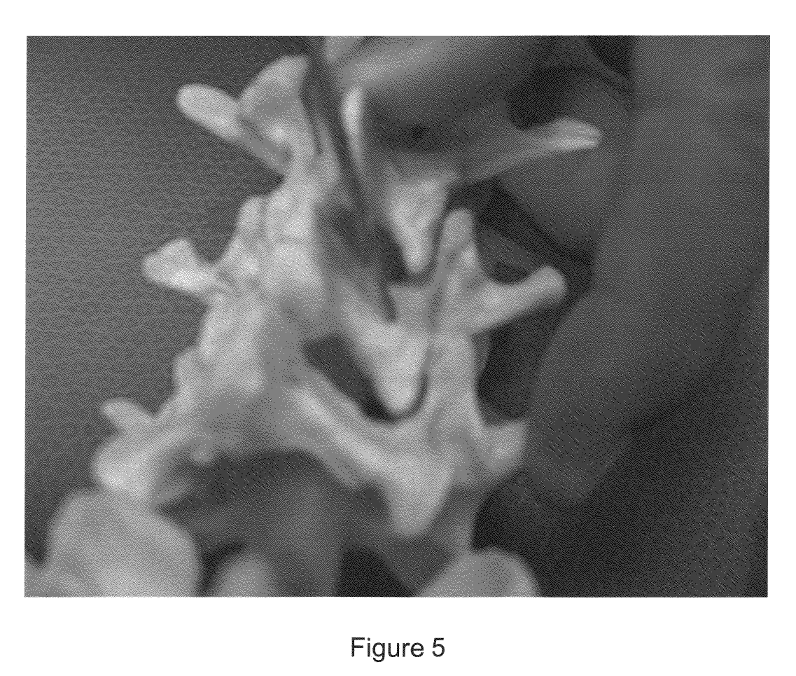

FIG. 6 shows a drill bit being placed for drilling into a vertebra in accordance with a preferred embodiment of the invention;

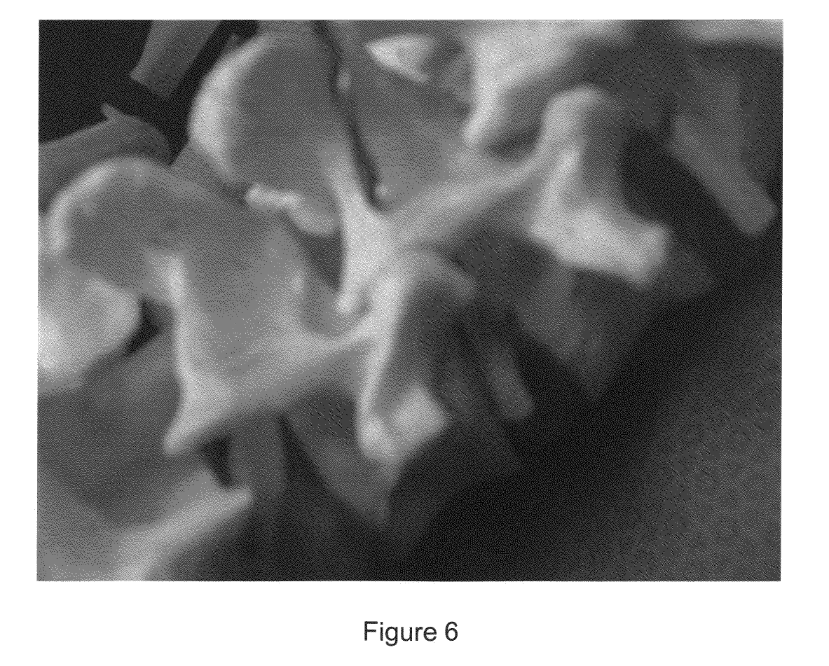

FIG. 7 shows a drill bit being placed for drilling into a vertebra in accordance with a preferred embodiment of the invention;

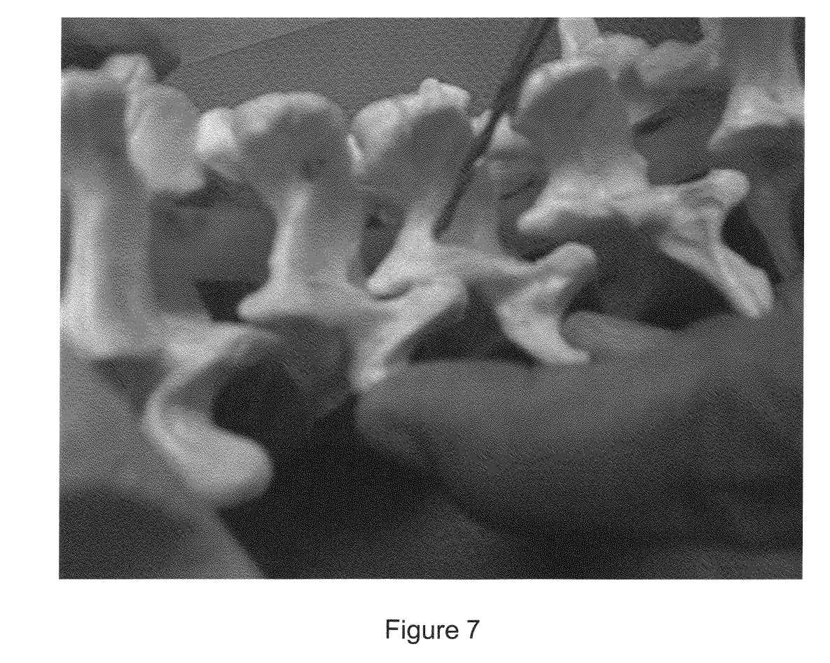

FIG. 8 shows a cannulated targeting device being used to guide a drill bit in accordance with a preferred embodiment of the invention;

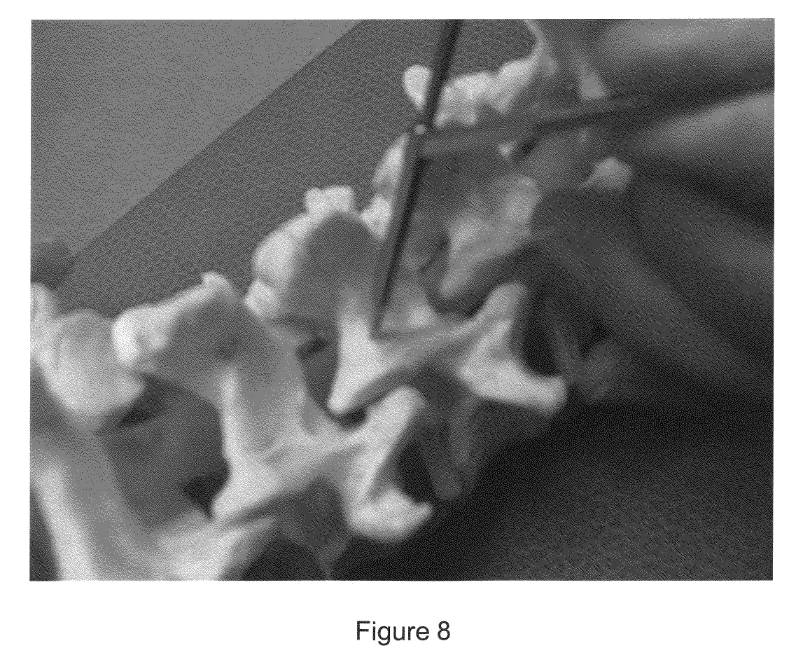

FIG. 9 shows a cannulated targeting device being used to guide a drill bit in accordance with a preferred embodiment of the invention;

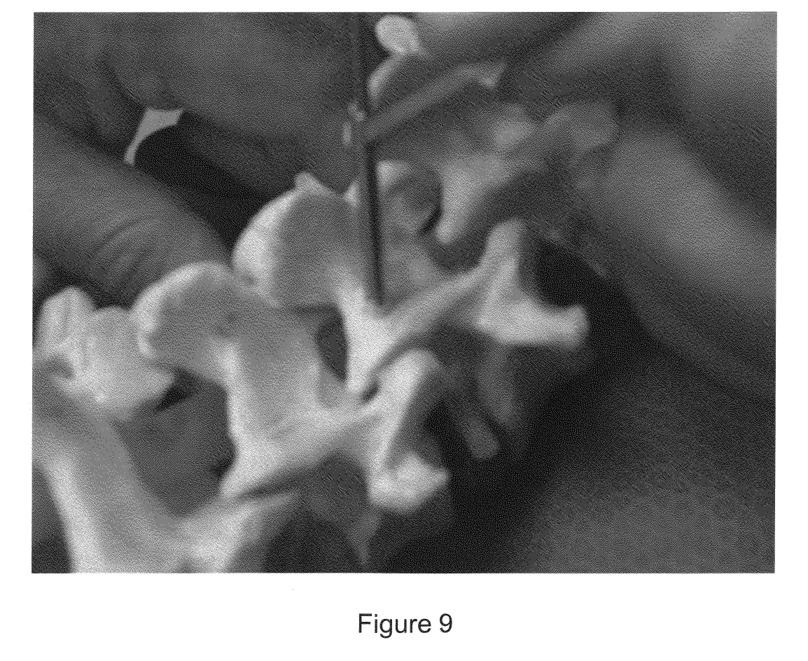

FIG. 10 shows a cannulated targeting device being used to guide a drill bit in accordance with a preferred embodiment of the invention;

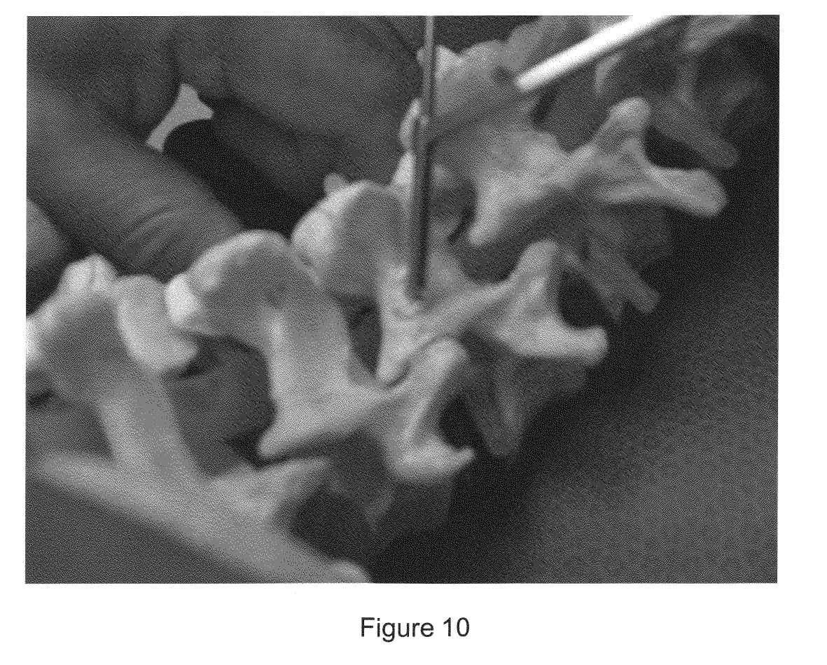

FIG. 11 shows a cannulated targeting device being used to guide a drill bit in accordance with a preferred embodiment of the invention;

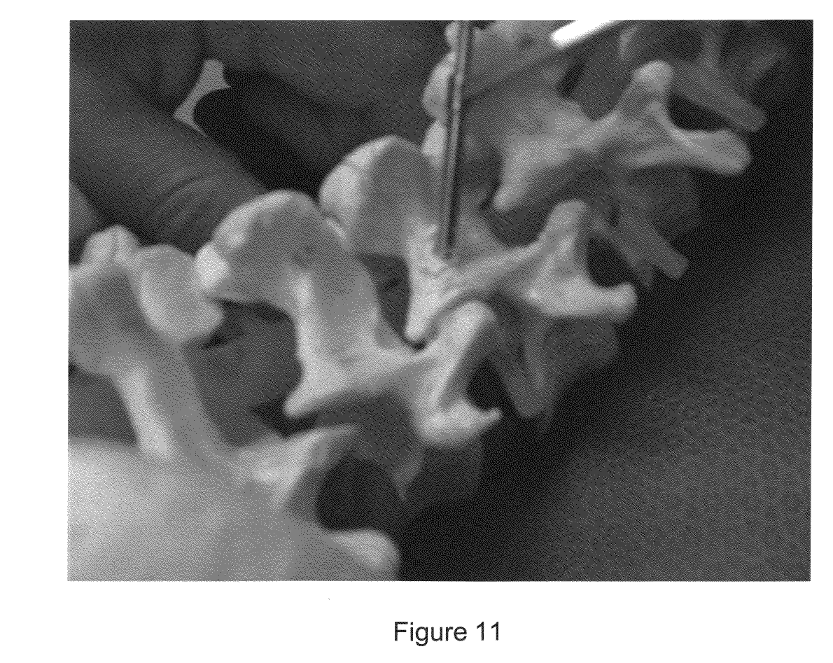

FIG. 12 shows a cannulated targeting device being used to guide a drill bit in accordance with a preferred embodiment of the invention;

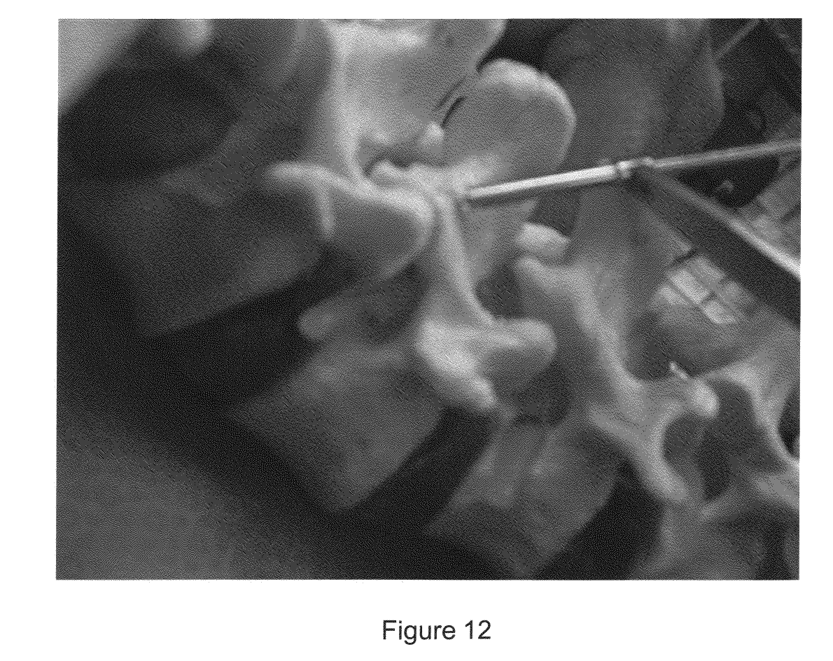

FIG. 13 shows a cannulated targeting device being used to guide a drill bit in accordance with a preferred embodiment of the invention;

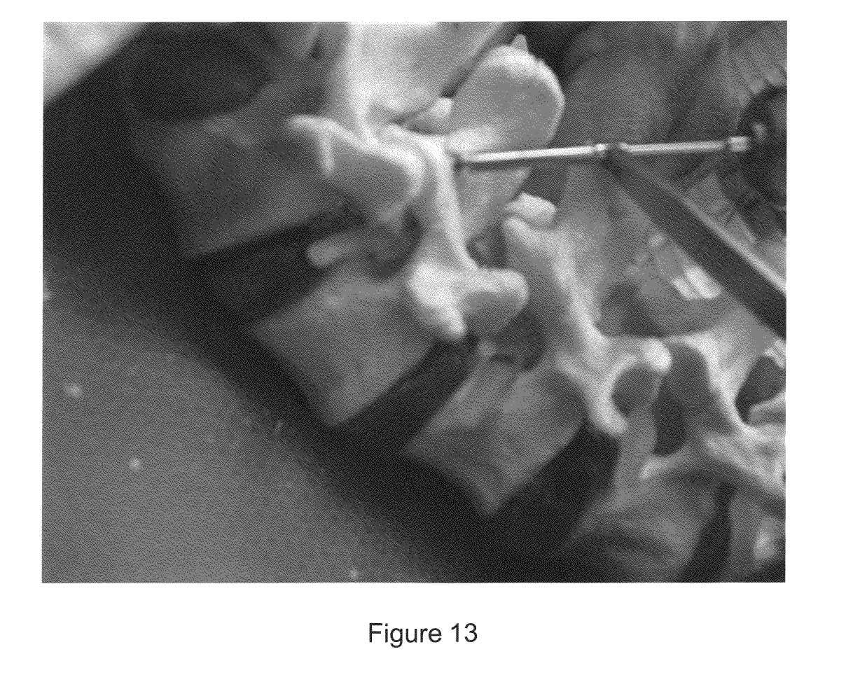

FIG. 14 shows a cannulated targeting device being used to guide a drill bit in accordance with a preferred embodiment of the invention;

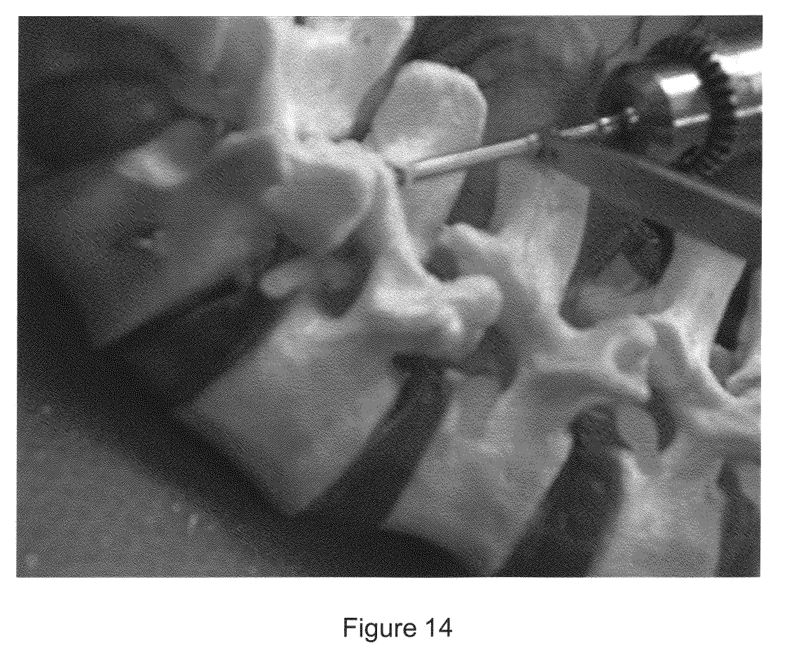

FIG. 15 shows a cannulated targeting device being used to guide a drill bit in accordance with a preferred embodiment of the invention;

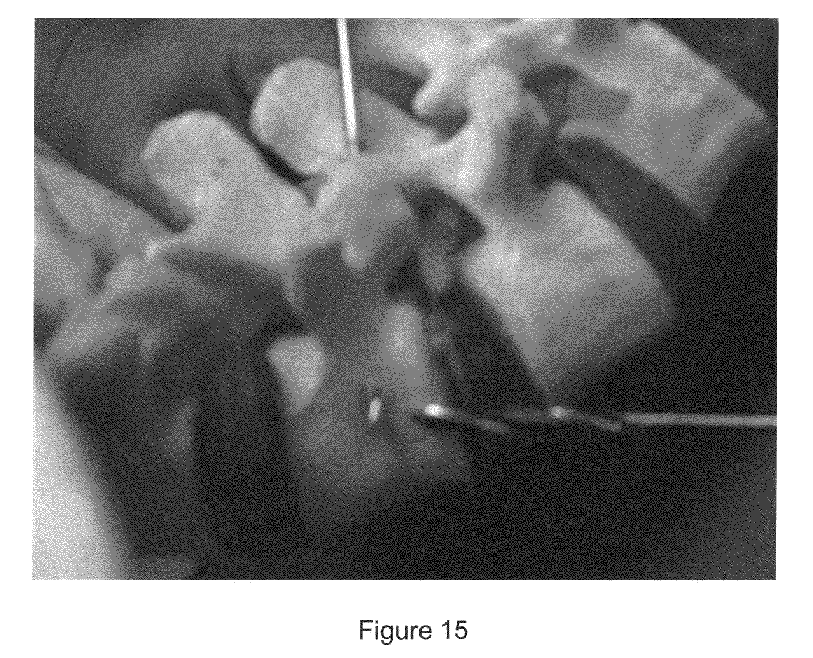

FIG. 16 shows a cannulated targeting device being used to guide a drill bit in accordance with a preferred embodiment of the invention;

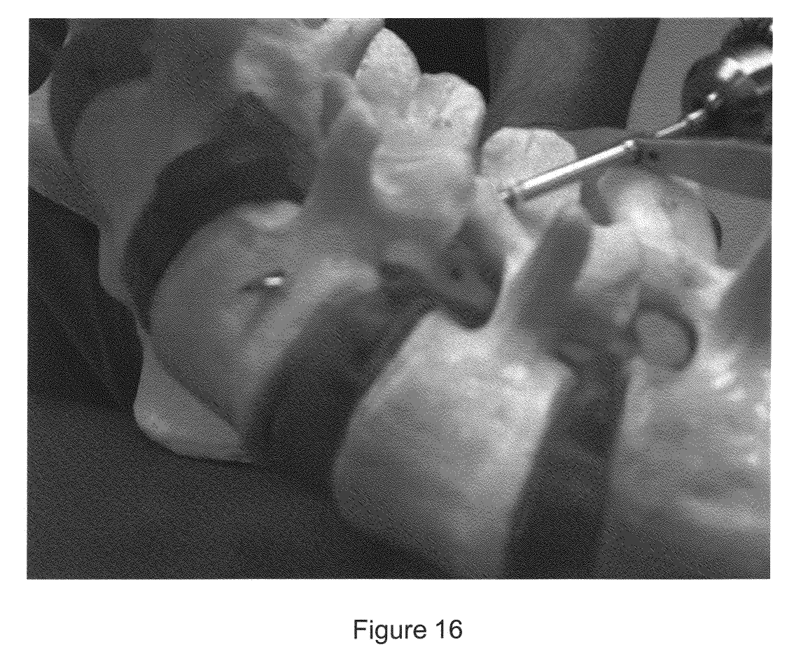

FIG. 17 shows a cannulated targeting device being used to guide a drill bit in accordance with a preferred embodiment of the invention;

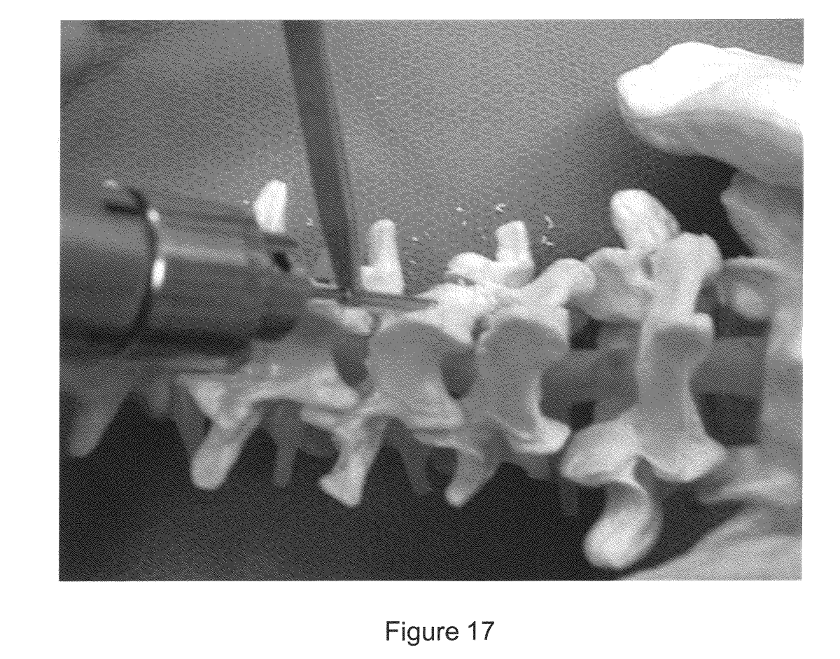

FIG. 18 shows a cannulated targeting device being used to guide a drill bit in accordance with a preferred embodiment of the invention;

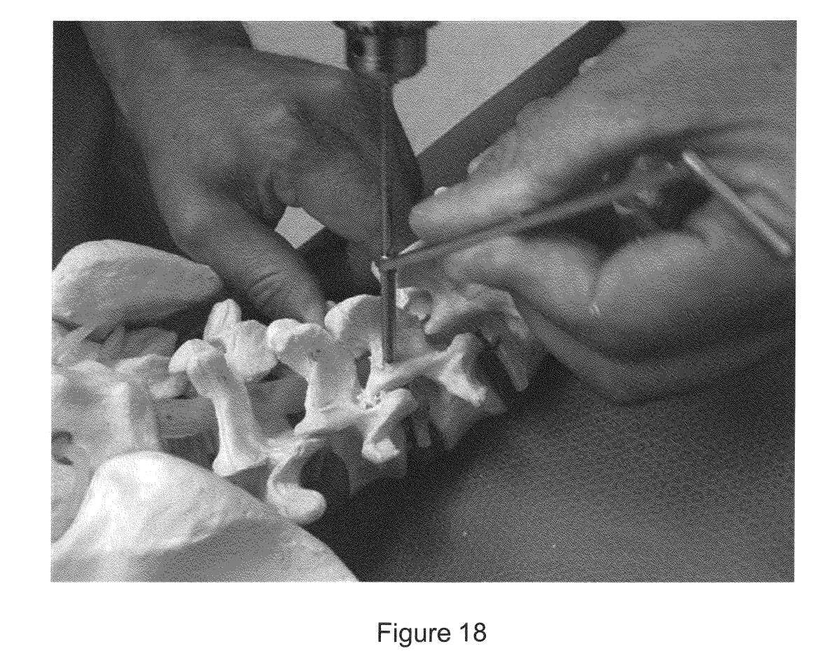

FIG. 19 shows a cannulated targeting device being used to guide a drill bit in accordance with a preferred embodiment of the invention;

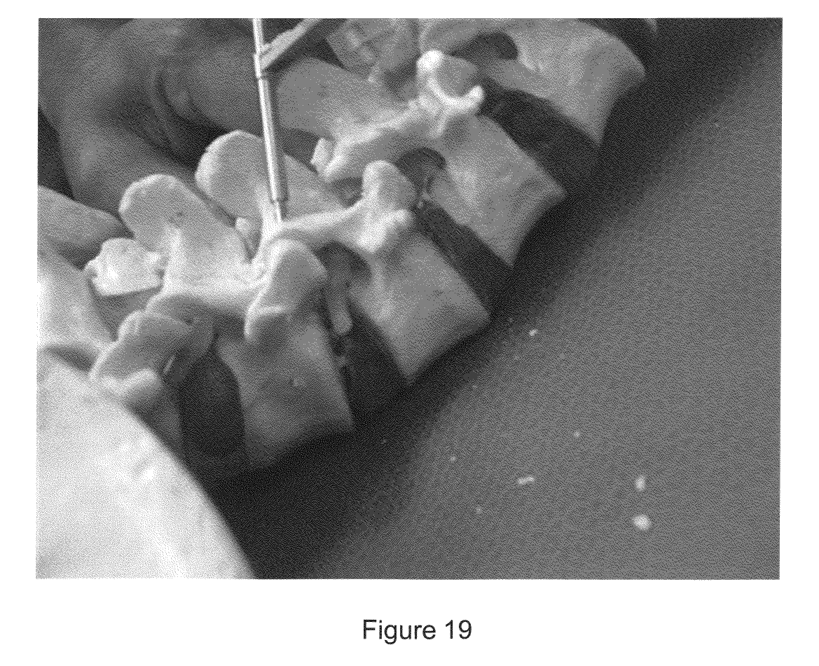

FIG. 20 shows a drill bit being placed for drilling into a vertebra in accordance with a preferred embodiment of the invention;

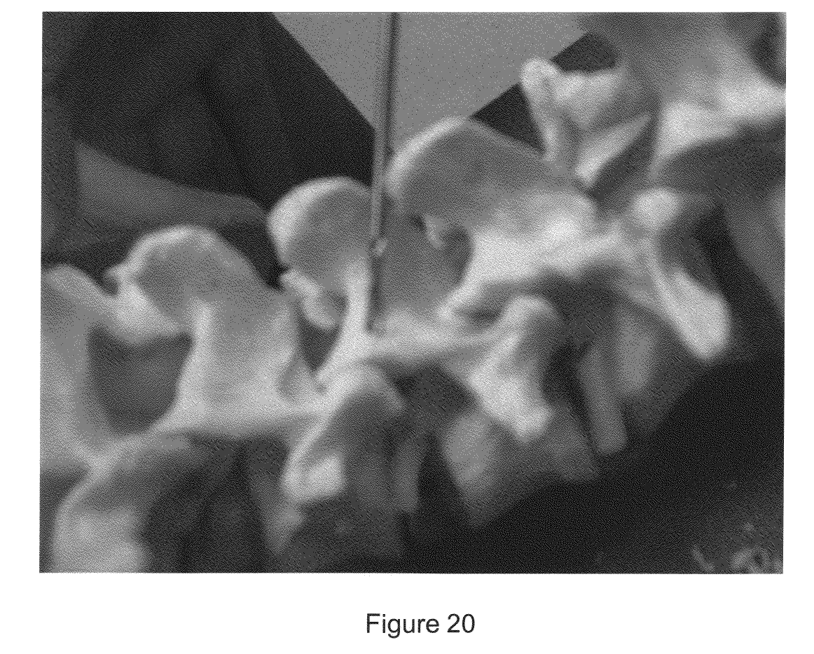

FIG. 21 shows an opening made in a vertebra in accordance with a preferred embodiment of the invention;

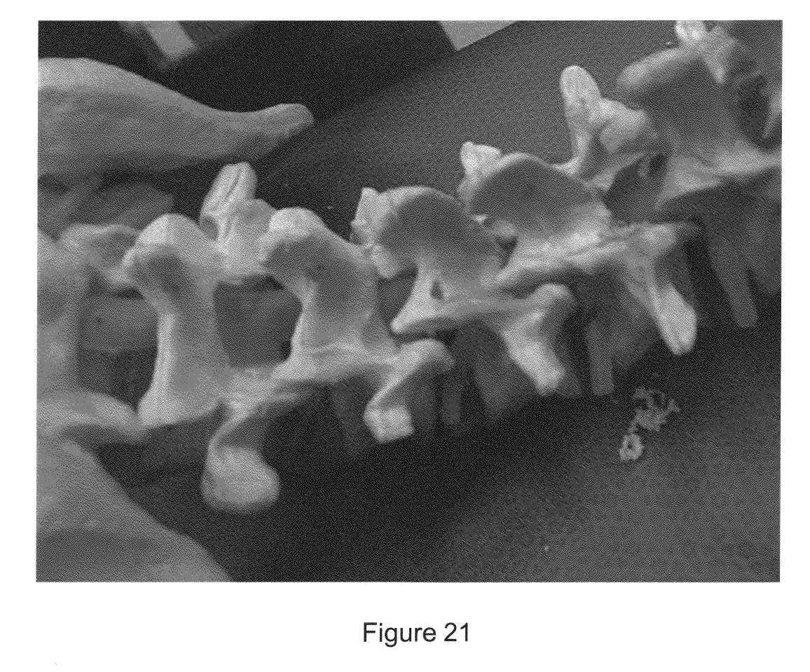

FIG. 22 shows an opening made in a vertebra in accordance with a preferred embodiment of the invention;

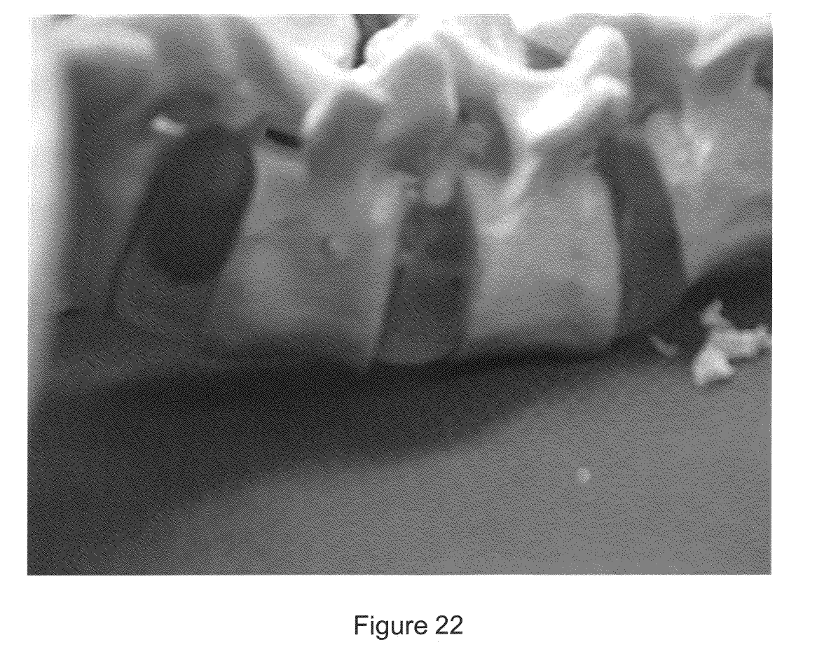

FIG. 23 shows an implant in accordance with a preferred embodiment of the invention;

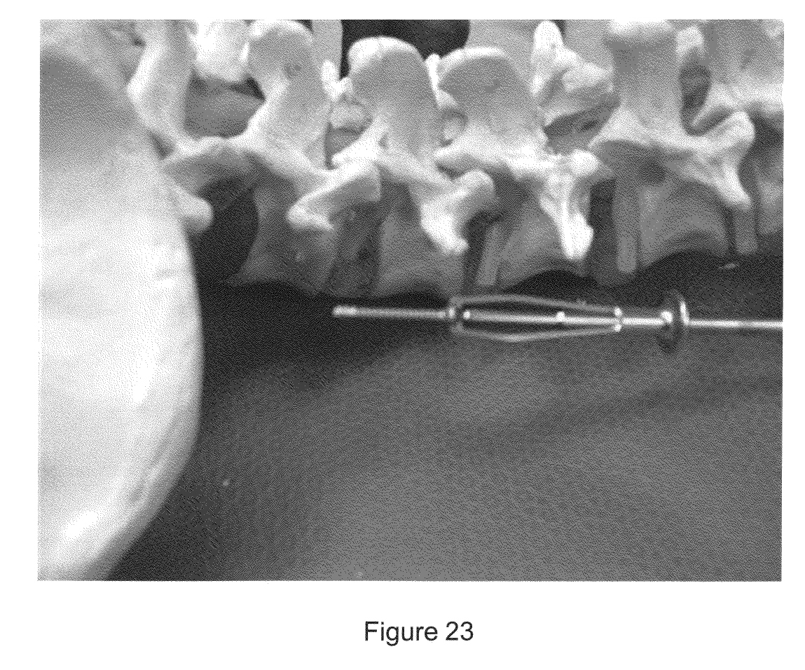

FIG. 24 shows an implant being placed in an opening in a vertebra in accordance with a preferred embodiment of the invention;

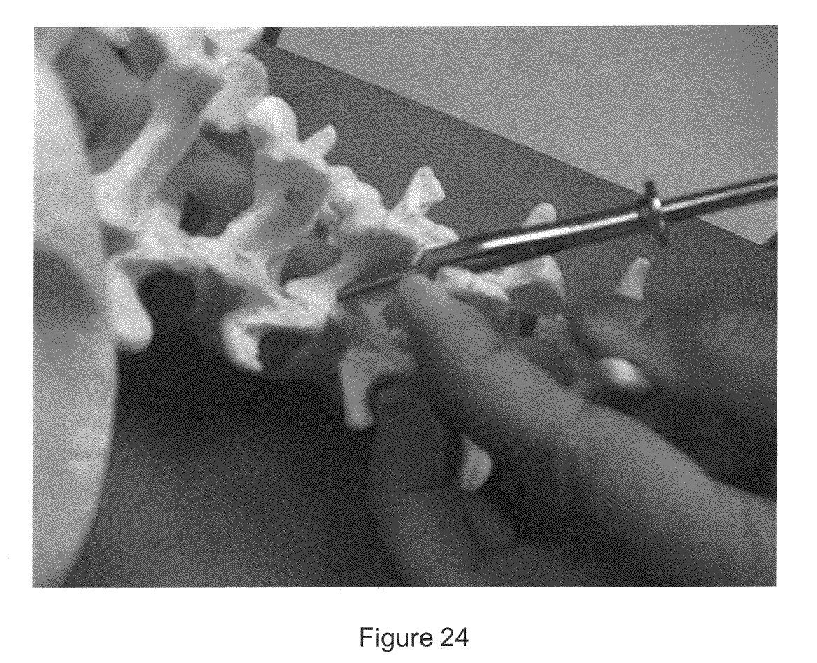

FIG. 25 shows an implant being placed in an opening in a vertebra in accordance with a preferred embodiment of the invention;

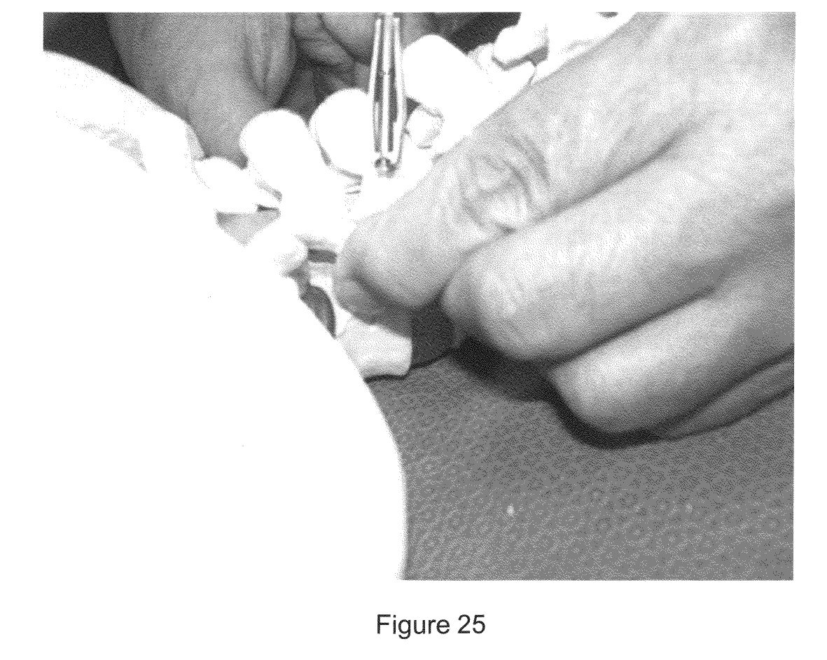

FIG. 26 shows an implant being placed in an opening in a vertebra in accordance with a preferred embodiment of the invention;

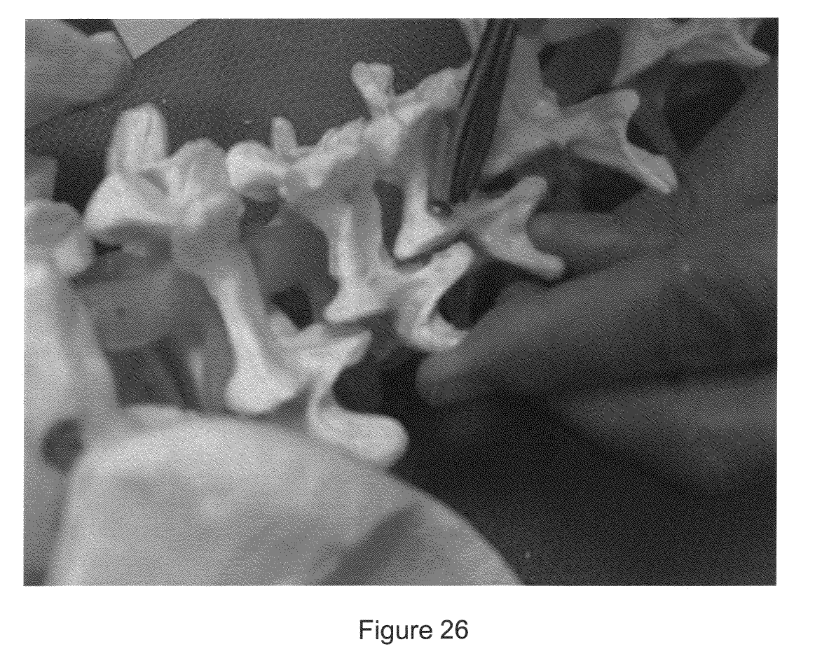

FIG. 27 shows an implant being placed in an opening in a vertebra in accordance with a preferred embodiment of the invention;

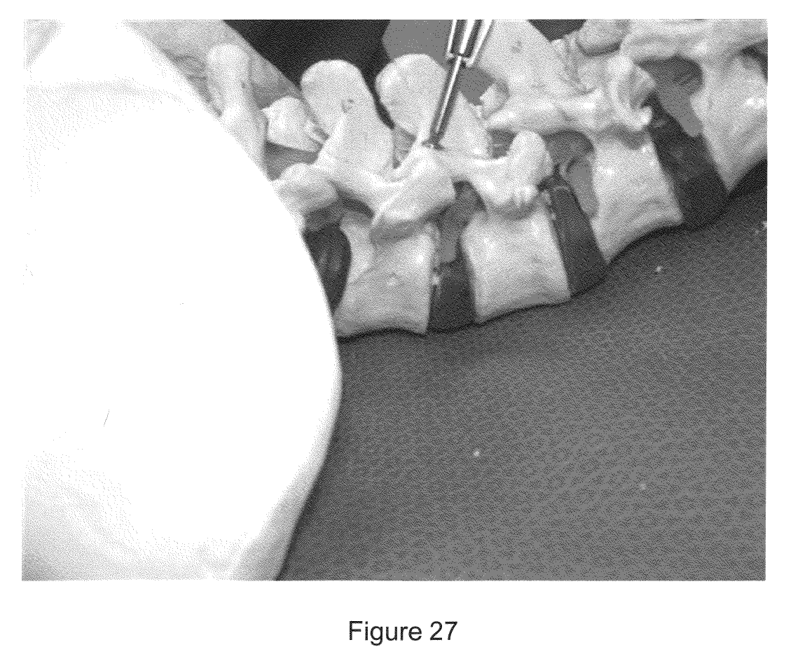

FIG. 28 shows an implant in place in a vertebra in accordance with a preferred embodiment of the invention;

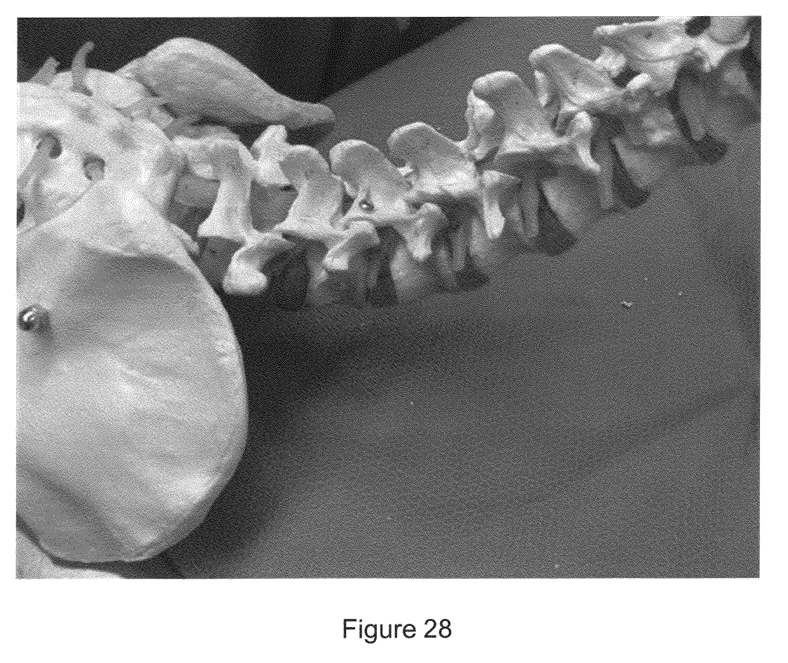

FIG. 29 shows an implant in place in a vertebra in accordance with a preferred embodiment of the invention; and

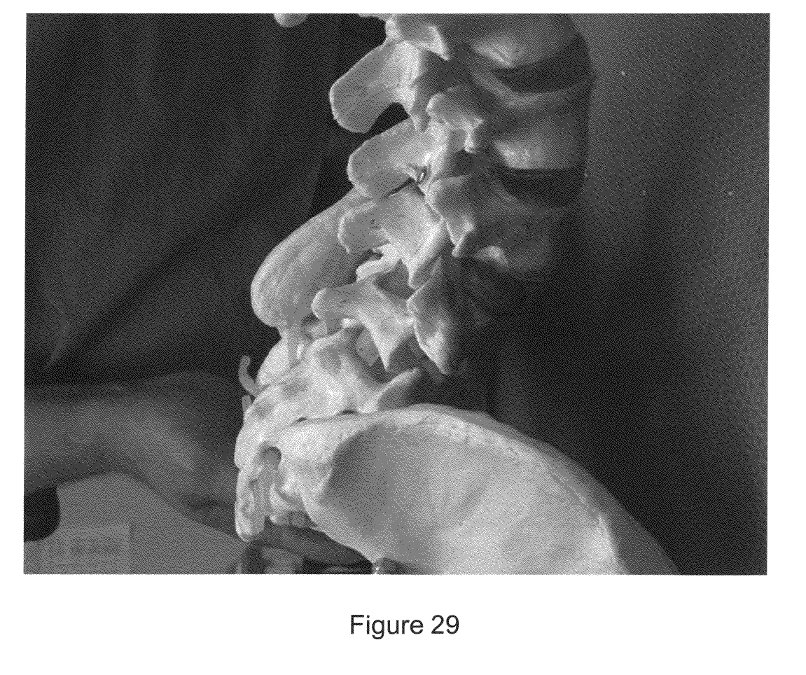

FIG. 30 shows an implant in place in a vertebra in accordance with a preferred embodiment of the invention.

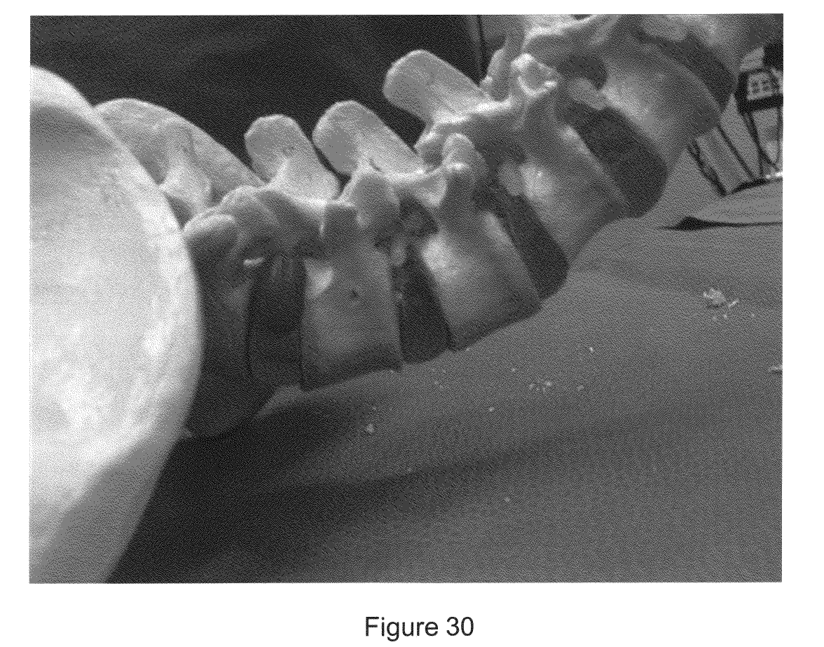

### DETAILED DESCRIPTION OF PREFERRED EMBODIMENTS

The invention relates generally to methods of spine fixation surgery and devices for use in spine fixation surgery. In a preferred embodiment, the invention comprises a method of performing spine fixation surgery. In another preferred embodiment, the invention comprises a cannulated targeting device which may be useful in the stabilization and direction of an implant during surgery. In yet another preferred embodiment, the invention comprises an implant for use during surgery.

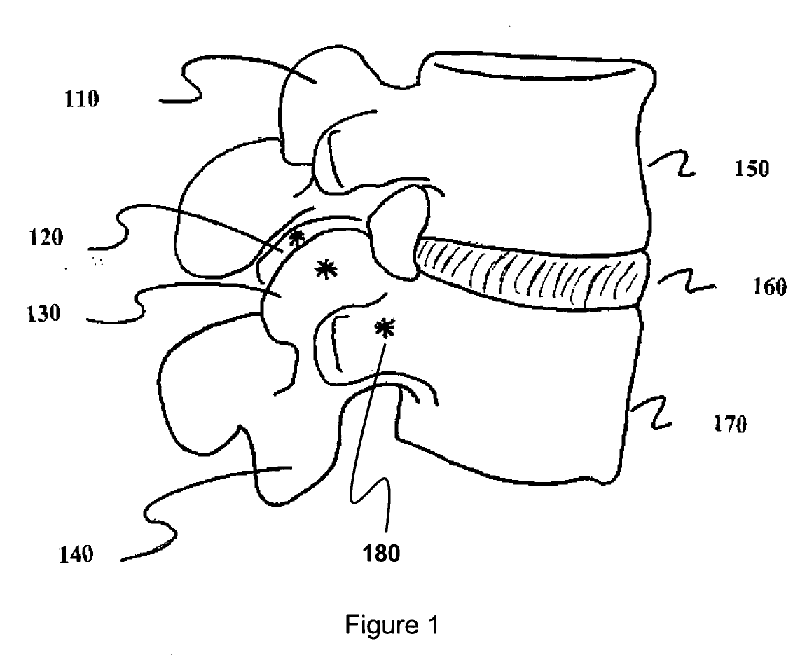

A preferred embodiment of the invention comprises a method of spine fixation surgery, generally related to FIG. 1. This method may further comprise using a cannulated targeting device to guide a drill bit through an upper vertebra (150) having a superior facet (110) and an inferior facet (120), and further through a lower vertebra (170) having a superior facet (130) and an inferior facet (140). The upper vertebra (150) and the lower vertebra (170) may be separated by a disk (160).

In one embodiment of the invention, the drill bit may progress from the superior facet optionally up to or minimally perforating a lateral pedicle wall to a maximal drilled depth of about 30 mm. In a preferred embodiment, the drill bit progresses from the inferior facet (120) of an upper vertebra (150) through a superior facet (130) of a lower vertebra (170), through a pedicle (180), and optionally through a lateral wall of the pedicle (180).

In this embodiment, an implant could be inserted into the opening created by the drill, and the implant would be subject to increased purchase when compared to an implant inserted into an opening which was differently aligned.

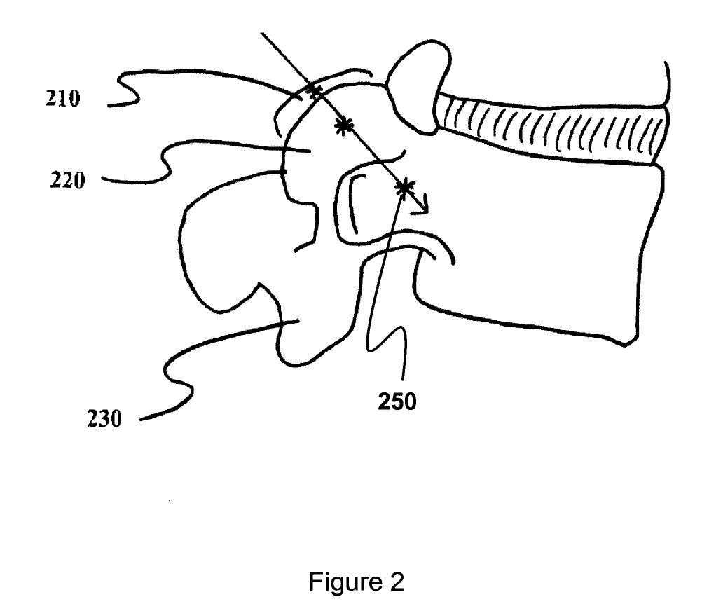

In a further preferred embodiment, generally related to FIG. 2, such a method may comprise the steps of: a) drilling into the inferior facet (210) of an upper vertebra at an angle (240) of approximately 20-30 degrees medial to lateral (traveling away from the midline of the body), and approximately 30-40 degrees caudal inclination (traveling away from the head of the body); and b) removing the drill bit to form an opening; and c) inserting an implant into the opening formed by the drill; and d) fixing the implant in the opening to secure the vertebrae. Following this trajectory may cause the drill bit to contact a superior facet (220) of a lower vertebra and optionally up to or minimally perforating the wall of a lateral pedicle (250) to a maximal drilled depth of about 30 mm in a preferred embodiment. The lower vertebra may also comprise an inferior facet (230).

In a preferred embodiment, a cannulated targeting device would be used to guide a drill bit at a standardized angle of approximately 20-30 degrees medial to lateral and 30-40 degrees inferior or caudal inclination such that the drill bit progressed from an inferior facet (120) of an upper vertebra through a superior facet (130) of a lower vertebra, through a pedicle (180), and optionally through the wall of a lateral pedicle (180). In this preferred embodiment, the cannulated targeting device would cause the drill bit to progress from a superior facet optionally up to or minimally perforating a lateral pedicle wall to a maximal drilled depth of about 30 mm, and prevent the drill bit from extending below the surface to be drilled more than about 30 mm.

In a highly preferred embodiment, a cannulated targeting device would be used to guide a drill bit at a standardized angle of approximately 25 degrees medial to lateral and 35 degrees inferior or caudal inclination such that the drill bit progressed from the inferior facet of an upper vertebra through a superior facet of a lower vertebra, through a pedicle, and optionally through a lateral pedicle wall. In this preferred embodiment, the cannulated targeting device would prevent the drill bit from extending below the surface to be drilled more than about 22-26 mm.

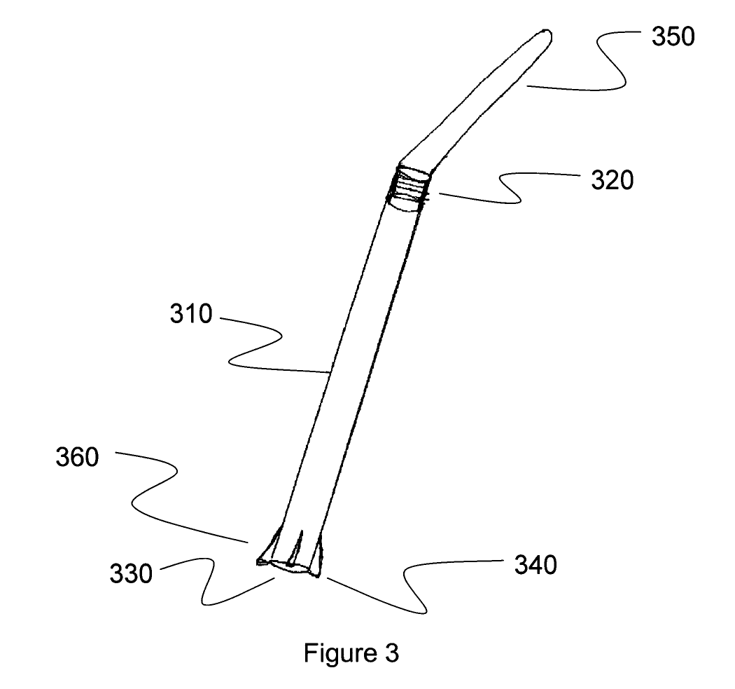

In a preferred embodiment, the current invention comprises a cannulated targeting device, generally related to FIG. 3. The device may comprise a tube (310) with at least a first opening (320) at one end and a second opening (330) at the opposite end, the two openings are capable of having a drill bit threaded through them such that the tube guides the drill bit at a particular angle relative to the surface to be drilled. This angle may be achieved by placing the face (340) of the second opening (330) against the surface to be drilled. The cannulated targeting device may further comprise a handle (350), shown here to be approximately at a 45 degree angle to the tube (310). The cannulated targeting device may further comprise a series of grooves or indicators at the first opening (320) for fixing the depth stop at a particular setting. The cannulated targeting device may also comprise a set of angled ridges (360), preferably four. These ridges may assist in fixing the angle of the device with respect to the surface. This angle may be an angle appropriate for drilling through an inferior facet of an upper vertebra, through a superior facet of a lower vertabra, and optionally up to or minimally perforating a lateral pedicle wall. The angle may be approximately 20-30 degrees medial to lateral (traveling away from the midline of the body), and approximately 30-40 degrees caudal inclination (traveling away from the head of the body), most preferably approximately 25 degrees medial to lateral, and 35 degrees inferior or caudal inclination, measured relative to the axis of the spine.

In a further embodiment, the cannulated targeting device may comprise a depth stop (320) which prevents the drill bit from being drilled more than a certain amount below the surface to be drilled. In a preferred embodiment, this amount of distance is not more than 30 mm, most preferably 22-26 mm.

In a preferred embodiment of the invention, the cannulated device is approximately 8.0 cm to 9.0 cm long, most preferably approximately 8.5 cm long, having a proximal end and a distal end. The cannulated device may comprise four angled ridges (360) along the distal end of the device. When present, the four angled ridges may be used in minimally invasive surgery in combination with imaging techniques, preferably fluoroscopic techniques, to target the device with respect to anterior/posterior (AP) pedicle view. In this embodiment, the cannulated device may be placed between about 2.5 mm and 3.5 mm above a superior pedicle ring and in line with a medial line of a pedicle wall on an inferior facet. A cutting device, preferably a drill bit, preferably approximately 11.5 cm in length, may be threaded through the cannulated targeting device. The proximal end of the cannulated targeting device may comprise a sleeve which can be extended along the length of the cannulated targeting device and fixed at set intervals at a depth stop in order to extend the length of the cannulated targeting device. In a preferred embodiment of the invention, the sleeve may be withdrawn at intervals of approximately 2 mm, up to an added length of approximately 10 mm. The canulated targeting device can be used to control the insertion of the cutting device to a depth of between 20-30 mm.

One of the novel aspects of the invention includes the angle of drilling and depth of the drilling, which results in increased purchase when the bones are held together by an implant. These angles are optimized for spinal fixation surgery in the lumbar area of the spine.

In a highly preferred embodiment, the cannulated targeting device is placed against an inferior facet of an upper vertebra, achieving a standardized angle of approximately 25 degrees medial to lateral and 35-40 degrees inferior or caudal inclination relative to the axis of the spine for drilling through a superior facet into a lateral pedicle wall. Maximal purchase is achieved in this approach.

A further embodiment of the invention comprises an implant which may be used in spine fixation surgery to secure an inferior facet of an upper vertebra to a superior facet of a lower vertebra and to a pedicle, and optionally project through a lateral pedicle wall.

In a preferred embodiment, the implant may be constructed of a biocompatible solid material, preferably titanium or polyetheretherketone (PEEK). The implant may be a shaft with a helical groove formed on its surface, and may be capable of advancing through a material at a first end of the implant when torque is applied to a second end of the implant. The implant may comprise threaded fastener component or a screw component. In a preferred embodiment, the implant may be capable of advancing through a material while creating a thread in the material (in the manner of a self-tapping screw). The implant may be capable of advancing through bone at a first end of the implant when torque is applied to a second end of the implant.

In a preferred embodiment, the threaded fastner component of the implant may be 3.5 mm to 4.75 mm in diameter, preferably 3.5 mm in diameter, and 8.5 cm in length with small blunt rocket fins angled in from approximately 2.5 mm on a compass 4 quadrant layout on the distal tip. This will allow targeting when looking down the barrel on an AP flouroscopic view with respect to the pedicle.

The size of the implant may be optimized within this range based on the size of a bone into which the implant is being inserted.

The implant may further include a plate component comprising a plate of a solid material, preferably disk-shaped, and preferably such that the threaded fastner component can be threaded through the plate component. The plate component may be a lock washer or a lock nut. In a preferred embodiment, the plate component would be capable of increasing the purchase of the threaded fastner component in the material into which it was being drilled. It is further contemplated that the plate component may be coated on one or more sides with a material which promotes bone formation, such as hydroxyapatite or bone morphogenic protein.

In a preferred embodiment, the implant may further comprise a lateral facet attachment extending laterally with respect to the length of the implant, preferably between 5 mm and 10 mm, and superiorly to with respect to the length of the implant, preferably approximately 5 mm, and inferiorly across the facet joint, approximately 5 mm. The lateral facet attachment may be composed of a malleable titanium mesh or biomaterial, and the surface, preferably the surface proximal to the implant, may be coated with hydroxyapatite or bone morphogenic carrier. This provides enhanced torsional stability and places biologic fusion material in direct apposition to facet joint for maximal fusion potential.

### EXAMPLE 1

A minimally invasive approach was used such that a cannulated device was inserted to the inferior facet of L4. Then, using flouro with targeting device, the proper insertion site was located. Radiographically, this was approximately 3 mm superior to the AP view of the superior ring of the L5 pedicle. The insertion site was located in line with the medial wall of the L5 pedicle. The proper trajectory as previously described was then drilled to 24 mm with a depth stop and 2 mm drill bit. A 3.5 mm proximal sliding hole was drilled with a 3.5 mm drill bit. The facet cartilage was removed and a 26 mm by 3.5 mm diameter titanium implant was inserted with optional lateral facet attachment or simple washer placed per surgeon bias.

Upon insertion of implant, AP and lateral flouro views was obtained to confirm proper implant placement. The cannulated device was removed and the incision closed.

### EXAMPLE 2

The standard open posterior approach to the lumbar spine was performed. Visualization of the inferior L4 facet and its articulation with the L5 superior facet was performed. With laminotomy the superior and medial wall of the L5 pedicle was palpated. The L5 transverse process was exposed for posterolateral fusion. Openly visualizing the known structures the cannulated device was placed and the proper trajectory drilled. A 26 mm by 2 mm tract was drilled with depth stop and then a 3.5 mm sliding hole was drilled. An implant 28 mm by 3.5 mm was then inserted after facet cartilage was removed and maximally compressed.

Optional facet fusion can be accomplished with lateral facet attachment containing pre-coated hydroxyapatite or bone morphogenic protein impregnated material on underside of implant. This aids in torsional rigidity and enhanced fusion incorporation of facet joint.

### Definitions

The definitions given below are to supplement the generally accepted definitions in the art.

The term “fixing” as used herein refers to any method of securing, making firm, or stationary, two parts relative to one another, as in spinal fixation surgery, wherein two vertebrae are fixed relative to one another.

The term “spinal fixation surgery” as used herein refers to a surgical method, open or minimally invasive, performed with the intent of fixing or making stationary two vertebrae relative to one another. This can be performed using implants to secure various elements of the vertebrae or by using various methods to encourage the growth of bone between the two vertebrae.

The term “fluoroscopic view” as used herein refers to an imaging technique to obtain real-time images of the internal structures of a patient, and may comprise the use of a fluoroscope, or a device consisting of an X-ray source and fluorescent screen, and may further comprise an X-ray image intensifier and a video camera or other recording device.

The term “depth stop” as used herein refers to a feature of a cannulated targeting device which impedes progress of a cutting device threaded through the device beyond a certain point, thereby limiting the depth that the cutting device may penetrate into tissue after passing through the device which is placed on the surface of the tissue. For example, a depth stop in one embodiment may comprise a moveable sleeve which fits around the length of a cannulated targeting device, and which is moveable along the long axis of the targeting device, and which is capable of being fixed at a certain point such that the length of the cannulated targeting device in addition to the sleeve which extends along the long axis of the device beyond the device itself is set at a certain length.

The terms “biocompatible material” or “bioabsorbable material” as used herein is any material which has the quality of not having toxic or injurious to tissue, or which may be reabsorbed due to normal biological function of the body, and may include titanium and polyetheretherketon (PEEK).

### REFERENCES CITED

The following references, to the extent that they provide exemplary procedural or other details supplementary to those set forth herein, are specifically incorporated herein by reference.

### U.S. PATENT DOCUMENTS

U.S. Pat. No. 5,000,165 issued on Mar. 19, 1991, with Watanabe listed as the inventor;
U.S. Pat. No. 5,584,887 issued on Dec. 17, 1996, with Kambin listed as the inventor;
U.S. Pat. No. 5,480,440 issued on Jan. 2, 1996, with Kambin listed as the inventor;
U.S. Pat. No. 5,643,259 issued on Jul. 1, 1997, with Sasso et al. listed as inventors;
U.S. Patent Publication No. 2005/0149021 published on Jul. 7, 2005, with Tozzi listed as the inventor;
U.S. Patent Publication No. 2006/0235388 published on Oct. 19, 2006, with Justis et al. listed as inventors;
U.S. Patent Publication No. 2008/0021473 published on Jan. 24, 2008, with Butler et al. listed as the inventors.

What is claimed:

1. A cannulated targeting device comprising:
a) a tube having a first opening at one end, a second opening at the opposite end, and a depth stop along the length of the tube; and
b) a face of the second opening capable of being placed against a surface, such that the tube achieves a standardized angle relative to the surface;
wherein the depth stop limits movement of a cutting device inserted through the tube such that the cutting device cannot move beyond a predetermined depth into the surface.

2. The cannulated targeting device of claim 1, wherein the fixed depth allows the cutting device to advance approximately 30 mm.

3. The cannulated targeting device of claim 2, wherein the fixed depth allows the cutting device to advance approximately 22-26 mm.

5. The cannulated targeting device of claim 1, wherein the surface onto which the face of the second opening is placed is a vertebra of a spine.

4. The cannulated targeting device of claim 5, wherein the standardized angle is approximately 20-30 degrees medial to lateral and 30-40 degrees caudal inclination relative to the axis of the spine.

5. The cannulated targeting device of claim 1, wherein the first opening of the cannulated targeting device is optimized to receive a drill bit.

6. The cannulated targeting device of claim 1, further comprising a handle attached to the tube.

7. An implant for fixing two vertebrae relative to one another, comprising:
a shaft constructed of a biocompatible or bioabsorabable solid material;
wherein the shaft comprises a helical groove formed on its lengthwise surface, such that the shaft is capable of advancing through a bone along its long axis at a first end of the shaft when torque is applied to a second end of the shaft; and
wherein the implant has been optimized for placement in lumbar vertebrae.

8. The implant of claim 7, wherein the implant has a diameter of 3.5 to 4.75 mm.

9. The implant of claim 7, wherein the implant has a length of 20-36 mm.

10. The implant of claim 7, wherein the solid material is titanium or polyetheretherketon (PEEK).

11. The implant of claim 7, wherein the implant is capable of advancing through a material and also creating a thread in the material.

12. The implant of claim 7, wherein the thread pitch and design of the implant are optimized for maximum purchase of bone.

13. The implant of claim 7, further comprising an attachment capable of contacting a lateral and superior portion of a superior facet and aiding in torsional rigidity.

14. The implant of claim 7, wherein the implant further comprises an attachment placed laterally relative to the long axis of the implant for increased purchase of lateral vertebral surface and increased torsional rigidity.

15. A method of fixing two vertebrae relative to one another, comprising:
connecting an inferior facet of an upper vertebra, a superior facet of a lower vertabra, and an interior portion of a lateral pedicle wall, using an implant.

16. The method of claim 15, wherein the lateral pedicle wall is perforated.

17. The method of claim 15, wherein the implant describes an angle in an upright spine of approximately 20-30 degrees medial to lateral and 30-40 degrees caudal inclination.

18. The method of claim 15, further comprising a preparation step of placing the implant in an opening formed by drilling through the bone of the upper and lower vertebrae.

19. The method of claim 18, wherein the preparation step further comprises aiming the drill using a cannulated targeting device, the cannulated targeting device comprising a tube having at least a first and a second opening; and a depth stop, and a face of the second opening capable of being placed against a surface, such that the tube achieves a standardized angle relative to the surface.

20. A method of fixing two vertebrae relative to one another, comprising the steps of:
a) creating an opening in an inferior facet of an upper vertebra at an angle of approximately 20-30 degrees medial to lateral, and approximately 30-40 degrees caudal inclination, and at a depth of no more than 30 mm;
b) inserting an implant into the opening; and
c) fixing the implant in the opening to secure the vertebrae.

21. A system for fixing two vertebrae relative to one another, comprising:
a cannulated targeting device; and
an implant;
wherein the cannulated targeting device comprises a tube having a first opening at one end, a second opening at the opposite end, and a depth stop along the length of the tube; and a face of the second opening capable of being placed against a surface, such that the tube achieves a standardized angle relative to the surface;
wherein the depth stop limits movement of a cutting device inserted through the tube such that the cutting device cannot move beyond a predetermined depth into the surface.
and wherein the implant comprises a shaft constructed of a biocompatible or bioabsorabable solid material, the shaft comprising a helical groove formed on its lengthwise surface, such that the shaft is capable of advancing through a bone along its long axis at a first end of the shaft when torque is applied to a second end of the shaft; and wherein the implant has been optimized for placement in lumbar vertebrae.
